(19) United States
(12) Patent Application Publication
(10) Pub. No.: US 2006/0008864 A1
(43) Pub. Date: Jan. 12, 2006

(54) Methods to measure compound specificity

(76) Inventors: Ashley Stuart Davis, Denver, CO (US); Kim Maria Middleton, Denver, CO (US)

Correspondence Address: CYTOSKELETON INC.;c/o ASHLEY DAVIS, 1830 S. ACOMA ST., DENVER, CO, 80223, US

(21) Appl. No.: 10/885,012

(22) Filed: Jul. 7, 2004

(51) Int. Cl. C12Q 1/02

(52) U.S. Cl. 435/29

(57) ABSTRACT

A system is presented that uses a microassay which measures the activity of a tubulin ligand on mammalian brain (standard) and cancer cell (pathogen) tubulin, and by calculation of the Tubulin Ligand Index a comparison can be made as to the potency of the compound towards cancer cell tubulin and the potential to predict the efficacy of using compounds for cancer treatments.

Compact Disc copies: This document is presented in electronic format on the duplicate CDs included with this application with a filename: CDS10-patent-09

Keywords: Tubulin Ligand Index, anti-cancer, ligands, specificity.

Abrreviations: TI=Therapeutic Index, IC50=Compound concentration for 50% inhibition of target protein activity, EC50=Compound concentration for 50% enhancement of target protein activity, TLI=Tubulin Ligand Index, CCT=Cancer Cell Tubulin, SPA=Scintillation Proximity Assay (Amersham Bioscience Inc. trademark). Mab=Monoclonal antibody. BBT=Bovine Brain Tubulin, DAPI=4′, 6-diamidino-2-phenylindole-HCl. ATCC—American Type Culture Collection.

### FIELD OF THE INVENTION

In the field of medicine there are several approaches for attempting to cure cancer, for example, surgery, radiotherapy and chemotherapy. Chemotherapy relies on the toxicity of chemicals that target cancer cells with more avidity than normal cells. Radiotherapy relies on the increased sensitivity of cancer cells to radioactivity compared to normal cells. Chemotherapy and radiotherapy have degrees of toxicity that vary with patient, cancer type, location, and dosing levels. There is a general index for the efficacy of a radio- or chemotherapeutic protocol, this is called the Therapeutic Index (TI) of a compound or protocol. In essence the TI is calculated by [compound concentration required for 50% toxicity of normal cells] divided by [compound concentration for 50% toxicity of cancer cells], the greater the value the more efficacious the drug. One major target for chemotherapy is the tubulin protein (4,5). It is well documented that chemotherapies targeting tubulin could benefit from improved TI values (for example refs. 6,7,8,9). In this invention, we present a method, the Tubulin Ligand Index (TLI) ratio determination, that can be used to identify compounds or improve existing ones with increased potency to cancer cell tubulin and hence improve the TI for chemotherapy. What is novel about this work is that it introduces a standardized assay format that can be used to measure the effects of a compound on any tubulin, In the past this has not been possible because there has not been such a combination of large scale production of specific tubulins, assay miniaturization, assay standardization and detailed comparative analysis between different sources of tubulin.

### BACKGROUND

In the late 1940's and 1960's two separate discoveries lead to purified compounds called vinblastine and paclitaxel that could be used to treat cancer. The vinblastine family of compounds were found to be the active components of a herbal “remedy” for treating cancer, which is isolated from Catharanthus roseus (formerly Vinca rosea). They are now widely used in treating childhood cancers like leukemia (1). Although they are potent compounds, patients develop neurotoxicity, neutropenia and liver toxicity, as well as eventually developing resistance to the compounds. In a similar vane, paclitaxel was discovered by isolating the active component of an extract of bark from the Canadian Yew tree (Taxus brevifolio) which showed increased toxicity on cancerous tissue culture cells. Paclitaxel is also a highly potent anti-cancer drug but with side effects similar to vinblastine (2,3).

Much later with developments in molecular science, the intracellular target protein for vinblastine and paclitaxel was found to be tubulin (4,5). This protein has a vital function in mitosis and cell division. During mitosis, the mitotic spindle is organized by tubulin and its polymerized derivative, microtubules, among other proteins. Because cancer cells have unregulated cell cycle progression, mitosis is a sensitive point in the cell cycle for tubulin ligands to arrest the cell division process and cause cell death by apoptosis.

Initial attempts to screen for tubulin ligands resulted in potent anti-tubulin compounds, however very few of these resulted in therapeutically active compounds i.e. they had low TI values. Cryptophycin 52 (6,7) and Dolstatin 10 and 15 (8,9) are examples that are very effective at targeting neuronal tubulin in vitro and show potent anti-cancer properties in tissue culture studies. Unfortunately these compounds were found to be highly toxic in clinical trials which were curtailed at Phase II's because of peripheral neurotoxicity (see references 10,11 for cryptophycin), and granulocytopenia (see reference 12 for dolastatin 10), and neutropenia (see references 13,14 for dolastatin 15). Likewise neurotoxicity issues with vinblastine and paclitaxel are key limitations to treat cancer (2,3).

Although tubulin is known to be the target of the first generation of potent anti-cancer compounds vinblastine and paclitaxel, the second generation of compounds did not gain much from this insight. A closer look at the tubulin used to generate the second generation of compounds finds that it may not represent the actual cancer cell target. Tubulin exists in six alpha- and six β-isotypes e.g. αβI, αβII, αβIII, αβIV, αβV, αβVI (15). Currently, there is controversy over whether these isotypes are significant for particular cellular processes or whether they can substitute for one another without any ill effects to cell physiology (Avila J, Microtubule Proteins, Ch 2, p 49-54). In cancer chemotherapy there is some evidence that tubulin isotypes are important determinants of the sensitivity of cancer cells to anti-tubulin chemotherapeutic agents, the main aspect that is found is that increased expression of βIII tubulin and perhaps βIV tubulins can result in resistance to paclitaxel (36,37), and increased βIII expression is also linked to less favorable prognosis in oligodendrogliomas (18). There is also a positive correlation between chemotherapy induced mutations of tubulin and the prognosis of particular cancers (e.g. U.S. Pat. No. 6,251,682). Neuronal tubulin (used in the second generation compound screens described above) is the standard protein for performing microtubule polymerization assays to determine compound potency against tubulin. This target protein is ideal for preliminary screens where large number of compounds have to be screened for initial tubulin binding activity. However there has been poor correlation between IC50 values determined from dose response curves on neuronal tubulin versus tissue culture or patient studies (for example, see references described above on Cryptophycin 52 and Dolstatin 10,15 [refs for basic research 6,7,8,9 compared to refs for clinical trials refs. 10,11,12,13,14]). This poor correlation may be due to different tubulin isotypes expressed in these cell types. Bovine neuronal tubulin has mainly αβII and αβIII tubulins, this is in contrast to HeLa cervical cancer cells which have mainly αβI and MCF-7 breast cancer cells which have mainly αβI and αβIV (FIG. 1). To define a more appropriate secondary screen we have focused on cancer cell line tubulin and tubulin isotypes to create a better target for anti-tumor drug design.

Treatment with current anti-cancer compounds leads to resistance due to multi-drug resistance (MDR) mechanisms and tubulin mutations (15,16,17). In the latter case, tubulin mutations have been introduced into cell lines which make suitable sources to isolate tubulin for biochemical studies and TLI measurements. The mutated forms make ideal drug discovery targets because they represent a late stage cancer development that may occur after initial chemotherapy has failed (15,18). However upto this time only very limited comparisons of drugs has been possible (e.g. refs 16, 26, 27, 28, 29) because the CCT is expensive to produce and a miniaturized system has not been available to measure IC50s.

Need for a Purified Protein Assay Based Drug Development System as Opposed to a Tissue Culture System.

Many drug screens rely on screening cell lines that are representative of the disease phenotype being targeted, for example, see U.S. Pat. No. 5,760,092 6,329,420 6,512,003 and 5,965,718. However a major caveat with tissue culture is that there are many metabolic changes when transplanting cells from the original host to in vitro. For example, in vivo tumors grow in necrotic and anoxic environments due to poor nutrient and oxygen limited conditions, in contrast tissue culture conditions have nutritious media and excess growth factors and oxygenation. It is not surprising then that there is limited correlation between drugs that have been identified from tissue culture inhibition assays and their clinical efficacy (see description of Cryptophycin and Dolstatin 10,15 compare refs 6 through 9 with refs 10 through 14). Clearly there are exceptions to this assertion e.g. paclitaxel and vinblastine are clearly potent in these assays and approved for clinical use.

Although many physiological and metabolic processes have changed enormously when a cancer cell is transposed from its host environment into tissue culture status there is evidence that the tubulin isotype composition does not change. For example Nicoletti et al. 2001 found that β1 tubulin was the most abundant β-tubulin isotype in 12 out of 12 human ovarian carcinoma xenografts (19). Another example is the HeLa cell line analyzed in this invention of which there are two cell lines (suspension and adherent) that were separated 49 years ago and they have undergone greater than 400 cell divisions since this time (refs 20,21 and ATCC website Cat# CCL2 and CCL 2.2), but they retain the same tubulin isotype composition (see FIG. 1). Therefore it is our view that by isolating the tissue culture tubulin and using that protein for directly targeting with compounds it will likely produce a more specific compound compared to tissue culture screening per se.

In summary, up to the time of this invention the analysis of anti-cancer drugs on CCT has not been well characterized or exploited in drug development. We have developed a system of analysis that objectively determines the activity of compounds on cancer cell line tubulin and hence opens the field for the development of new potent chemotherapeutic agents.

### DETAILED DESCRIPTION OF THE INVENTION

The Need for a Micro-Assay

Because cancer cell tubulin (CCT) is much less abundant than neuronal tubulin and the raw material (tissue cultured cells) is much more expensive than brain tissue from an abattoir, CCT is much more expensive to isolate. In this regard we have developed a fluorescent assay that is capable of being miniaturized which effectively puts this target protein in the realm of screening technology.

In addition another ligand binding assay exists for measuring the affinity of compound for a particular ligand site on tubulin. In 2000, Tahir et al. developed the Scintillation Proximity Assay technology (Amersham Bioscience) into a tubulin ligand assay (22). SPA technology requires a close association between a solid phase scintillant (the beads) and the radio-ligand for a signal to be emitted and subsequently detected. Biotinylated tubulin is the reagent that brings the radio-ligand and the scintillant into close association. If the radio-ligand is tritiated colchicine for example then the amplitude of signal is proportional to the number of colchicine binding sites that are occupied by this radio ligand. Cytoskeleton Inc. introduced biotinylated CCT in 2003, which allows the researcher to use only 1 ug of protein per SPA assay, rather than 24 ug in the polymerization assay, again making savings in economy for screening purposes. The caveat is that only one site can be probed at one time, but this is a realistic alternative to measure TLI values compared to the polymerization assay described in detail in this application.

Measuring Tubulin Polymerization in vitro.

There are two basic tubulin polymerization assays these are the absorbance based assay (23) and the fluorescence based assay (24). The absorbance assays actually measures light scatter, the more scatter the less light reaches the detector, this assay is less sensitive to short microtubule formation which is under-represented in the initial stages of polymerization. The fluorescence based assay uses a fluorophore (DAPI) that binds preferentially to the microtubule conformation of microtubules compared to the un-polymerized tubulin monomers.

Both assay formats create a similar kinetic polymerization profile (see FIG. 2). There is a nucleation phase that represents the aggregation of tubulin monomers into protofilament or ring-like structures. These nucleation centers create a framework for more specific aggregation that elongates the protofilaments and closes a cylinder called the microtubule. Further microtubule elongation is represented by the polymerization phase (see FIG. 2), then when there is an equilibrium between the monomer and microtubule the phase is called steady state. There are basically two types of tubulin ligand, first those that bind to the monomer and inhibit its polymerization like vinblastine, and secondly compounds that enhance polymerization like paclitaxel. Enhancing compounds decrease the nucleation phase, increase the rate of polymerization and increase the extent of polymerization i.e. a higher steady state value. In the case of monomer binding compounds, which inhibit polymerization, all of these phases are oppositely affected. There is a comprehensive review of tubulin polymerization in Chapter 2 of Microtubule Proteins, (25) and a review of drug activities in Chapter 4 of the same book.

The Need for Standardizing the Tubulin Polymerization Assay for Comparative Analysis

Tubulins isolated from different origins are well known to have different kinetic assembly properties (i.e. polymerization rates and extents differ) (e.g. Davis et al. 1993). FIGS. 3 and 4 show the effects of glycerol on tubulin polymerization. Glycerol is viewed as a general enhancing agent with no specific site of action on the tubulin molecule. Instead glycerol is thought to enhance polymerization by the exclusion of water from the surrounds of the microtubule (26). All tubulins so far analyzed are similarly affected by glycerol, in the sense that glycerol enhances polymerization, thus by using a suitably high amount of glycerol i.e. 20%, one can promote tubulin polymerization to a similar extent independent of the source of tubulin.

This is shown in FIG. 4 where the microtubule component is compared to the tubulin monomer component by SDS-PAGE analysis of pellet and supernatant fractions of centrifuged samples. Microtubules are large complexes which pellet when centrifuged at greater than 50,000× g, in comparison to the monomers which stay in the supernatant. FIG. 4C indicates equivalent amounts of microtubule polymer from three different tubulin isolates when polymerized under identical conditions with 20% glycerol. The supernatants are likewise similar as expected because the tubulins are all titrated to 2 mg/ml before use.

Thus 20% glycerol can be used as a standardizing agent that allows a constant amount of polymerization to occur in a reaction containing 2 mg/ml tubulin.

The Tubulin Ligand Index System

The Tubulin Ligand Index system is different from previous analysis of tubulin isotypes and cancer tubulin sources (e.g. 16, 26,27,28,29) because it creates a measurement or value that is able to be cross referenced between experiments. By comparing the IC50 or EC50 values between two tubulins, one of pathogenic origin and one of normal tissue origin, an exact ratio of specificity can be determined. The key step in standardizing this measurement is to use glycerol as a polymerization enhancing agent for example in the “20% v/v glycerol control reaction”. Glycerol can be viewed as a global enhancer of polymerization in contrast to tubulin ligands that target one site on the molecule (33). Thus using 20% v/v glycerol we can standardize every assay no matter what source of tubulin or molecule being tested.

Another key aspect of the Tubulin Ligand Index is the constant concentration of tubulin, optimally 2 mg/ml tubulin protein concentration, but other concentrations can be used as well.

Another key aspect of the Tubulin Ligand Index is the use of a control or standard source of tubulin which sets the standard response which is used to compare the pathogen or cancer cell tubulin. A suitable standard tubulin would vary depending on the medical requirement, for example a neuronal tubulin would be a good standard to use for developing anti-cancer compounds of which family member compounds cause neurotoxicity as a side effect e.g. paclitaxel analogs. Another example would be any mammalian tubulin when deriving anti-fungal agents with fungal tubulin. Thus identifying one pathogen's tubulin source i.e. fungal or cancer cells, and a standard tissue which is negatively affected by known anti-tubulin drugs, the drug developer can compare IC50s using the TLI and determine which compound is likely to be more selective at killing the pathogen.

A powerful aspect of the TLI system is its robustness to errors in drug concentration. Errors in weighing and molarity calculations are a reality in drug discovery where large numbers of compounds may be screened. Because the TLI value is a ratio measured from the same stock solution of compound then the absolute concentration of drug is not important because it is canceled out in the calculation (see later for more on this aspect).

A review of Current Patent Status

A USPTO search for the keyword “tubulin” in patent abstracts on Apr. 11th 2004 gave 70 hits. Four of these described assays relevant to this work. U.S. Pat. No. 6,500,405 describes an assay that measures ligand binding to tubulin by its inverse relationship to a reactive species that modifies un-liganded tubulin. U.S. Pat. No. 6,346,389 uses a spin-down assay for measuring the “Survivin” protein (protein's name) in association with microtubules, if a ligand disrupts this interaction it will be detected by a reduction in the amount of pelleted Survivin protein. U.S. Pat. No. 6,306,615 uses antibodies raised to modified tubulins to measure the amount of liganded tubulin in patients, which is a diagnostic but not a drug development screening tool. U.S. Pat. No. 5,972,640 describes a cell based assay that measures the potency of drugs, possibly tubulin ligands, in comparison to a control cell line that has a cell cycle defect. All of the above assays are either cell or patient based or they are based on neuronal tubulin. There is no consideration of purified cancer cell tubulin or the composition of tubulin isotypes.

Additionally, many of the patents are concerned with identifying anti-cancer drugs based on growth inhibition of tissue culture cells followed by characterization with purified neuronal tubulin. This has been the standard drug discovery process for anti-cancer tubulin ligands over the past twenty years. For example see U.S. Pat. No. 5,760,092 6,329,420 6,512,003 and 5,965,718. None of these patents contain the use of purified cancer cell tubulin in comparison to neuronal tubulin as a primary or secondary screen.

A Review of Current Non-Patent Publications

There are several articles citing the importance of tubulin isotypes or tubulin source for the potency of tubulin ligands (e.g. 16, 26,27,28,29). These publications describe either spin-down assays that measure microtubule concentration or absorbance assays which measures light scatter by microtubules (similar to the absorbance data in FIG. 7). Small amounts of tubulin were isolated and only one or two compounds were characterized. This type of analysis is performed at the very latest stages of drug development where many decisions have already been made as to which compounds are suitable for FDA applications. The paucity of data concerning specialized tubulins is due to the expensive and difficult nature of purifying the specialized tubulins i.e. those other than neuronal tubulin. The current invention circumvents these hurdles by combining a microassay with a method of standardization that allows historical data to be compared for many years hence. In particular this assay format is suitable for drug screening at an earlier stage during drug development such that CCT specific drugs can be identified in primary screens which otherwise might be discarded.

Timeline of the Invention

In 1994 Cytoskeleton Inc. realized a requirement for a more specific tubulin for cancer drug development applications, this was exemplified by the introduction of purified HeLa cell tubulin to the company's product line in 1995 (see 1995 Catalog). Since this time there has been steady progress in developing better purification regimes, multiple sources of tubulin, robust assays and better data analysis. The development progress of this invention has been discussed at our booth in the American Association for Cancer Research meetings since April 2000.

### DESCRIPTION OF DRAWINGS

FIG. 1: Table of tubulin isotypes comparing Neuronal with HeLa and MCF-7 tubulins.

Data were collected from Banerjee and Luduena 1993 (28) and the present invention. Methods of quantitation are described in FIG. 3 description.

FIG. 2: Typical tubulin polymerization assay.

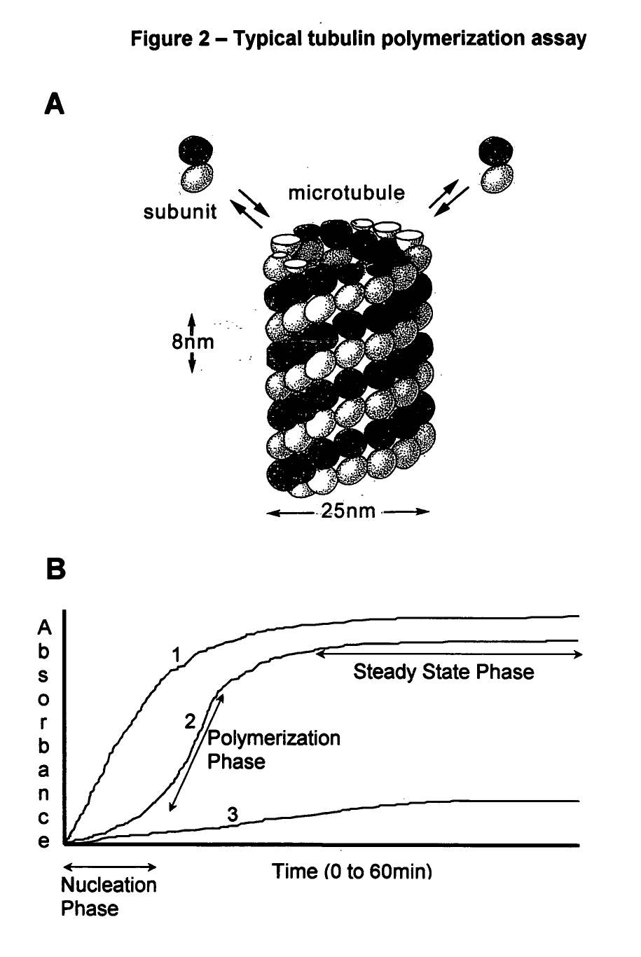

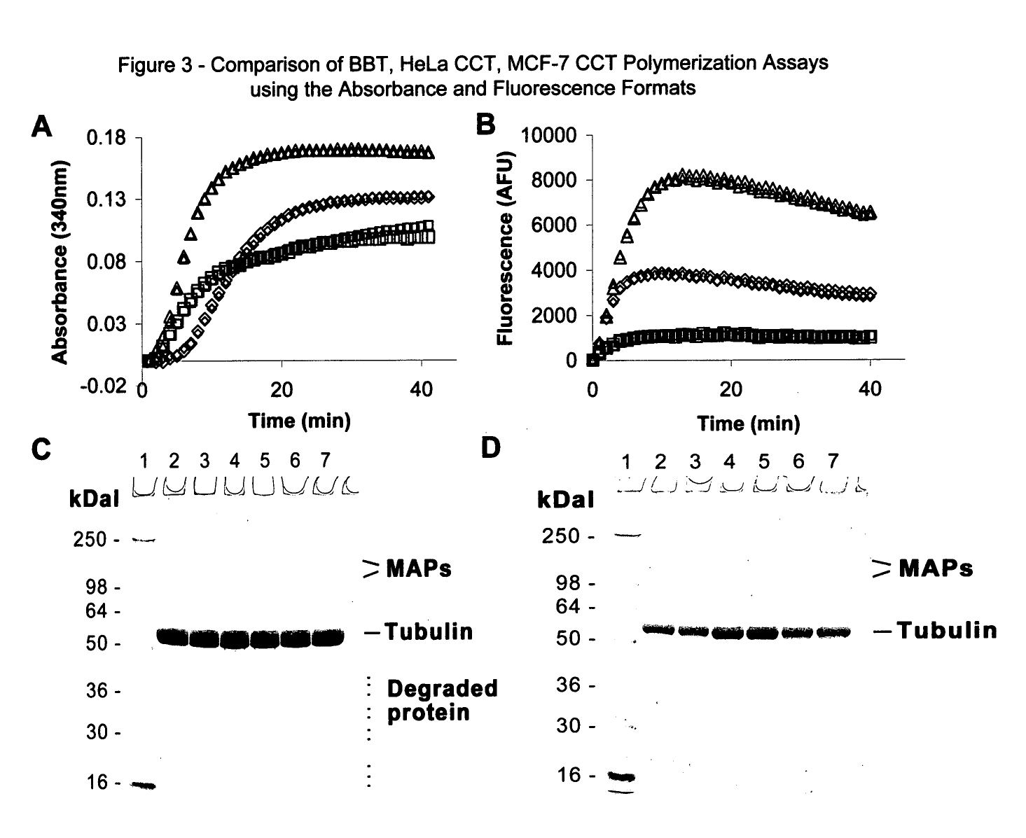

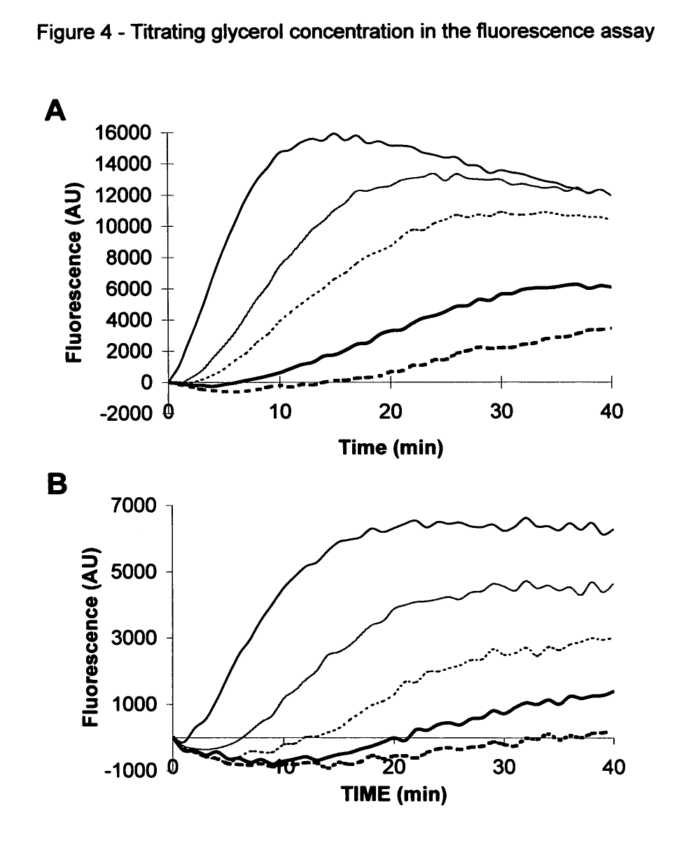

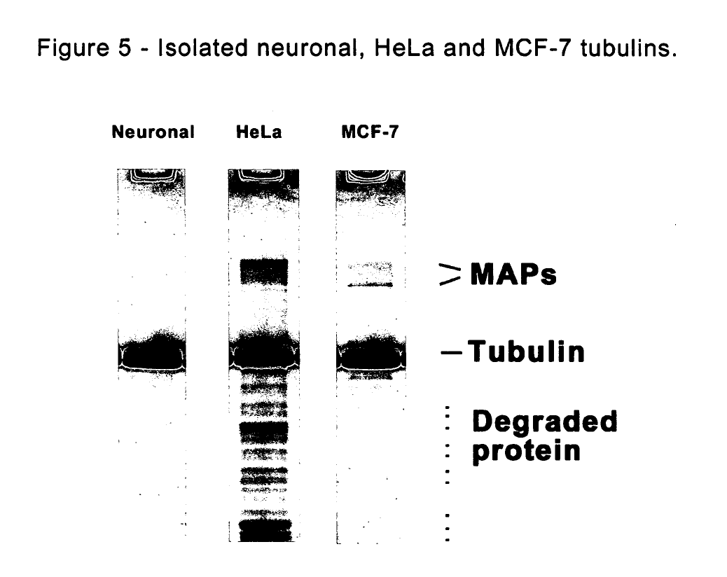

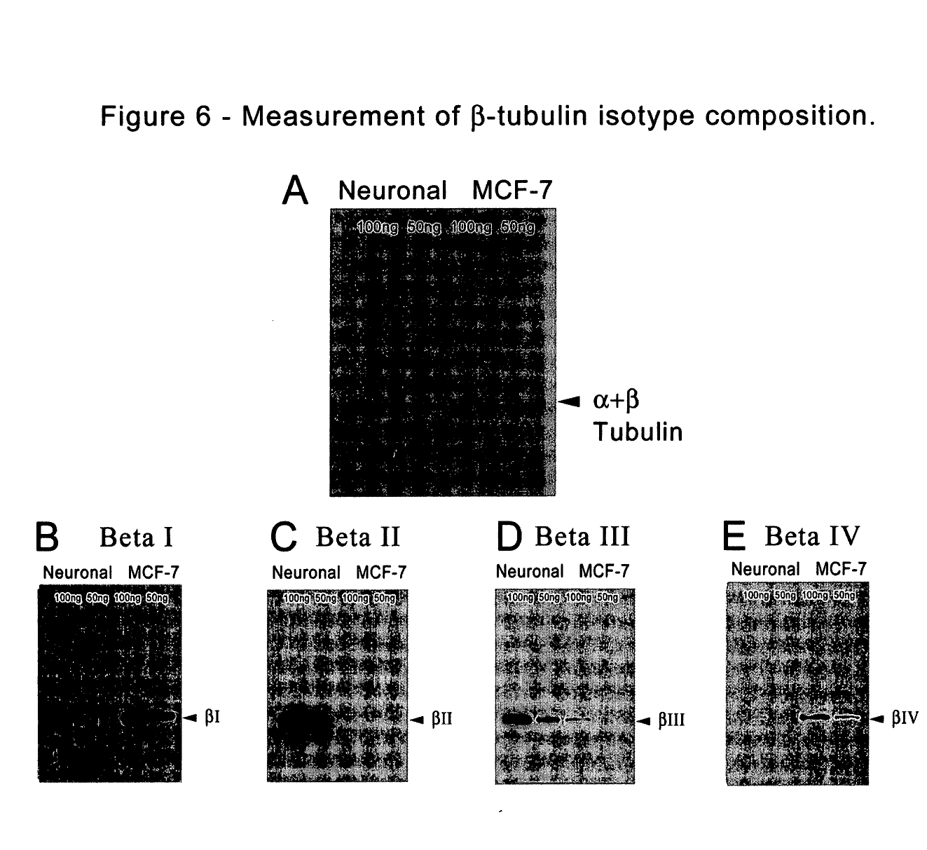

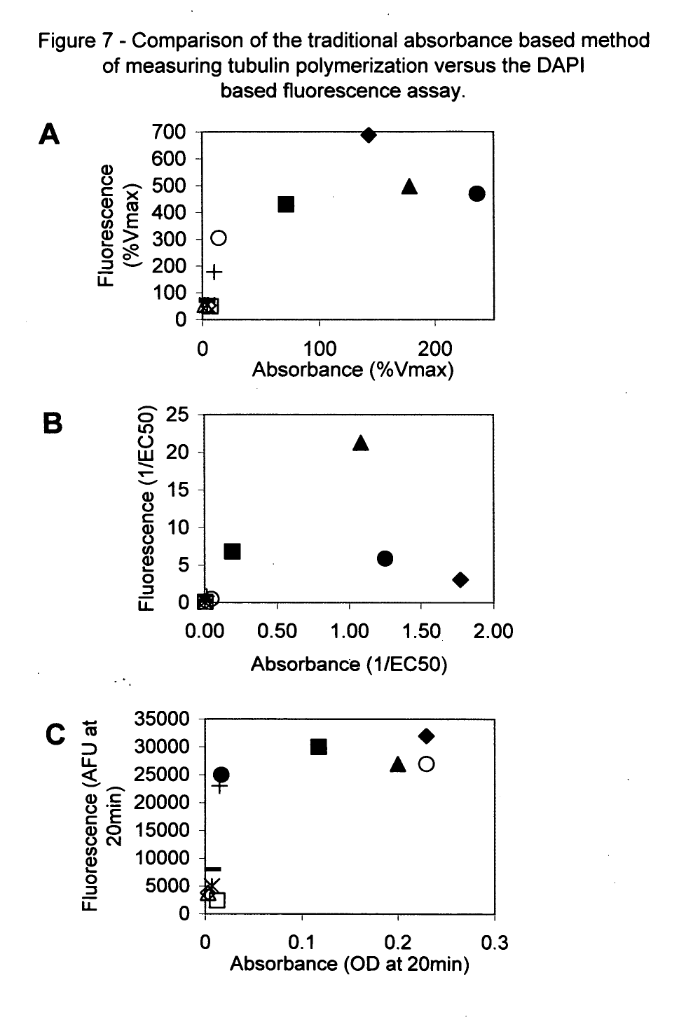

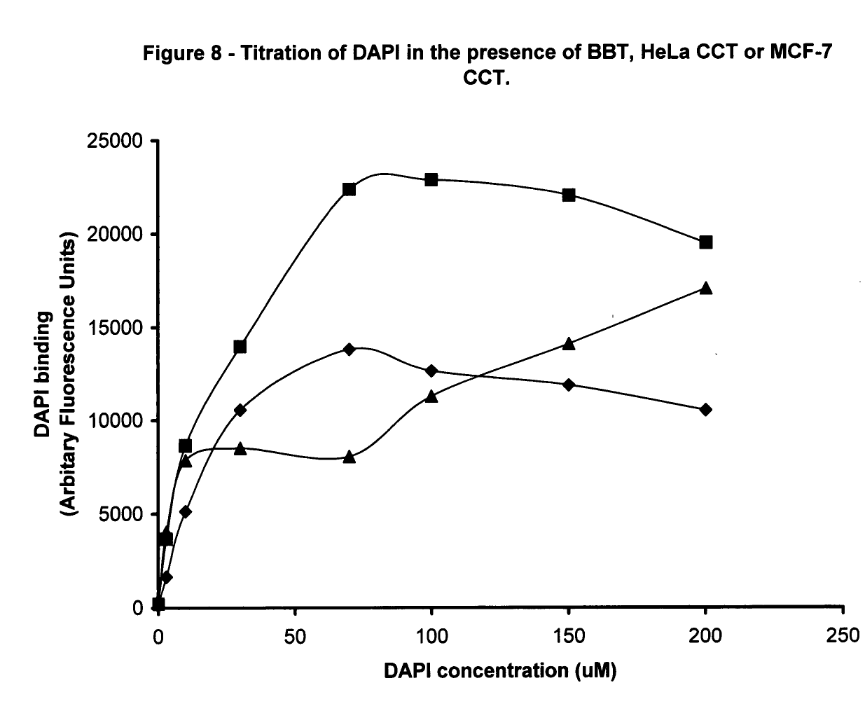

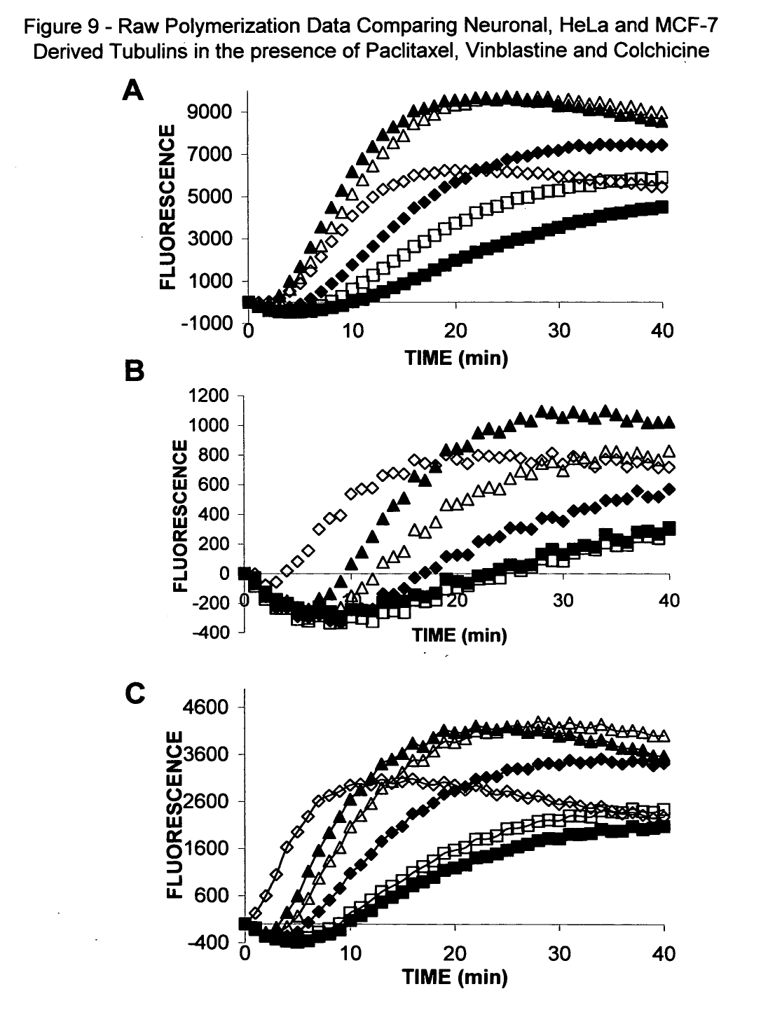

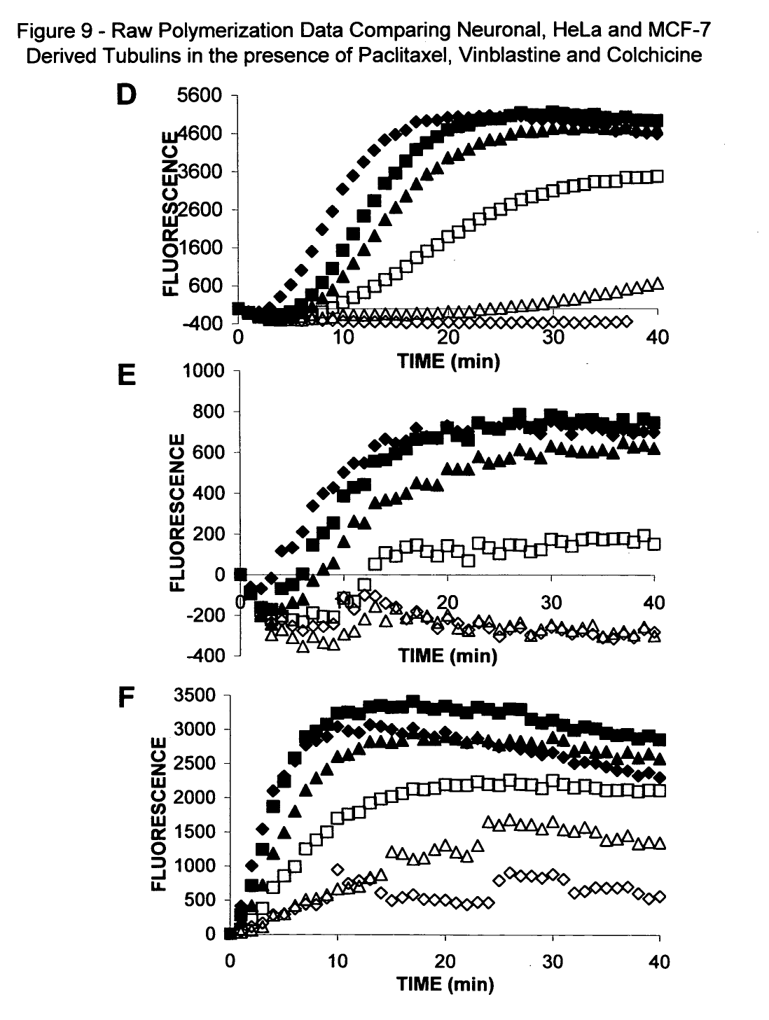

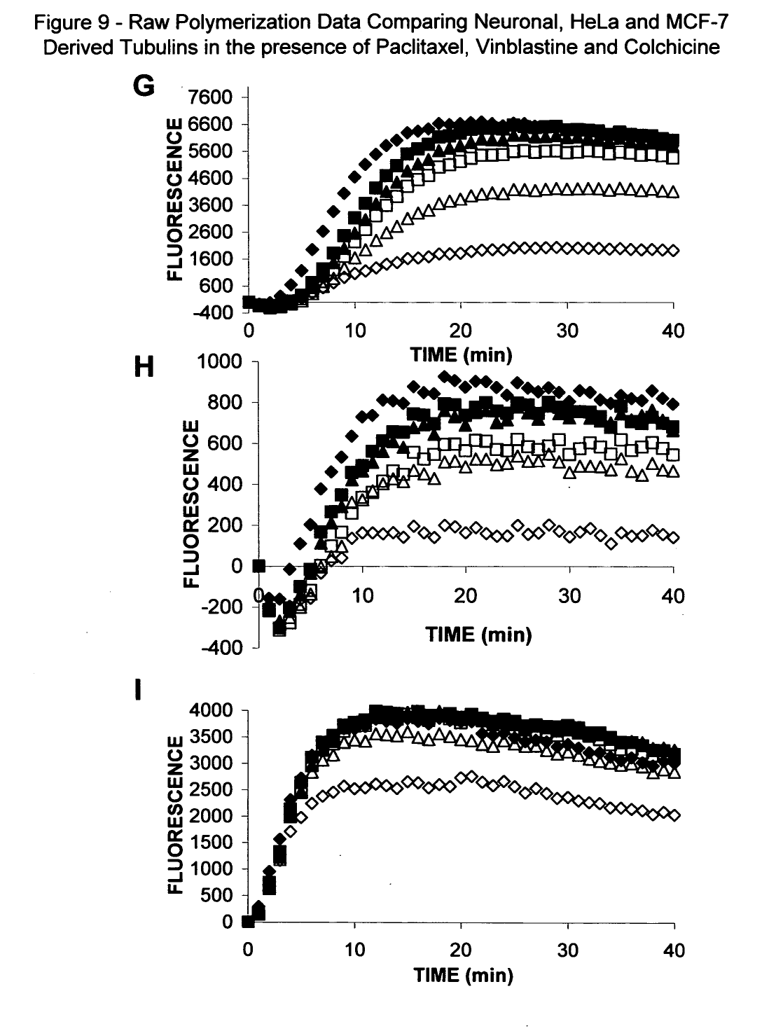

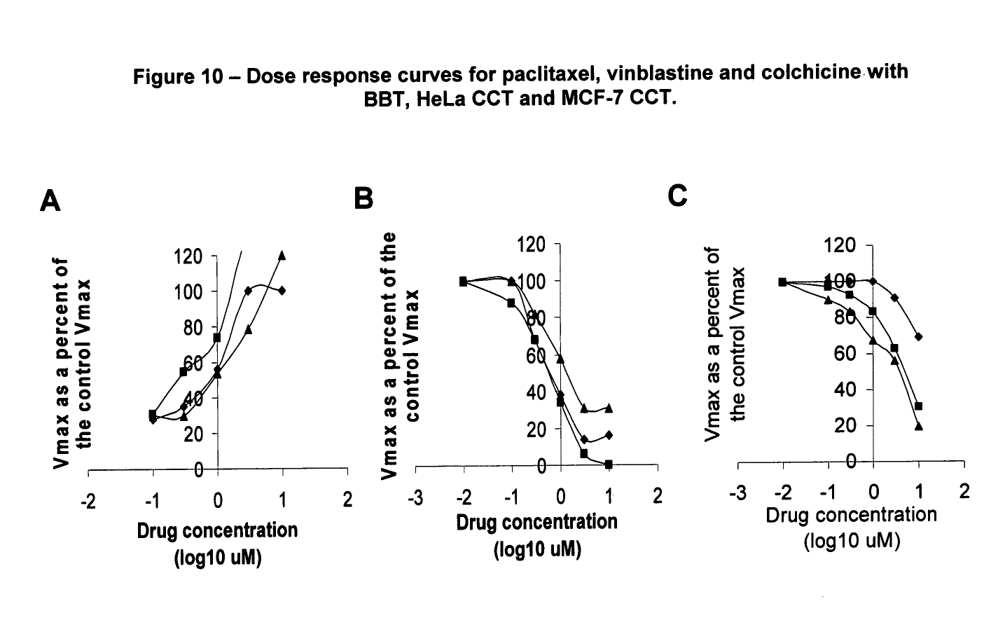

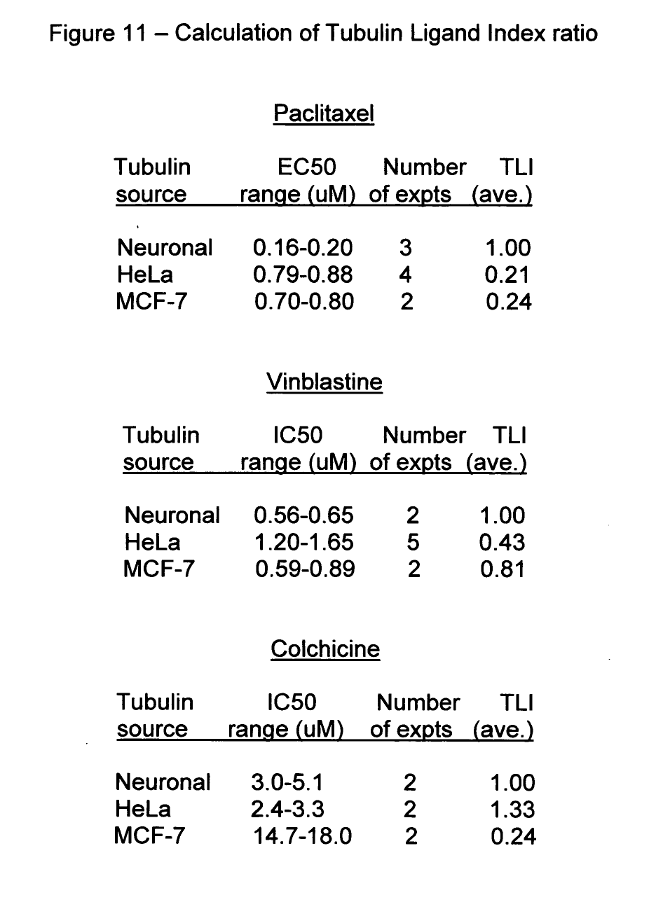

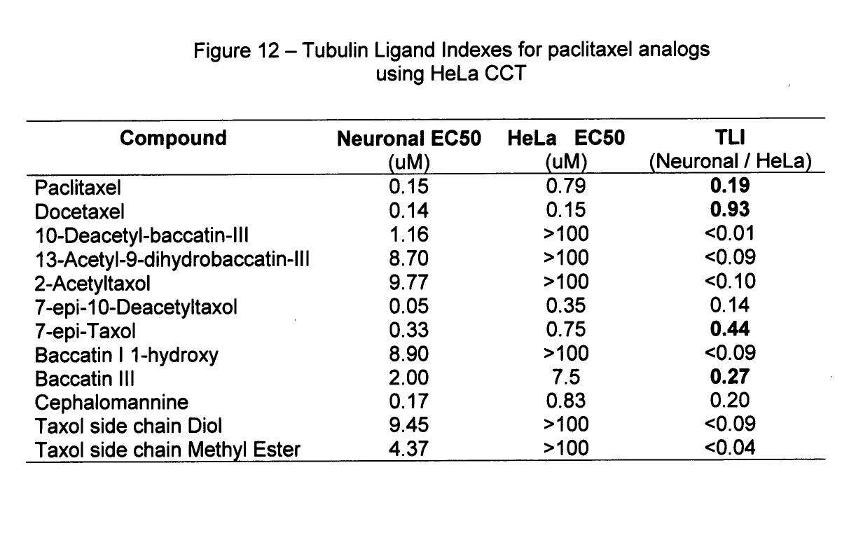

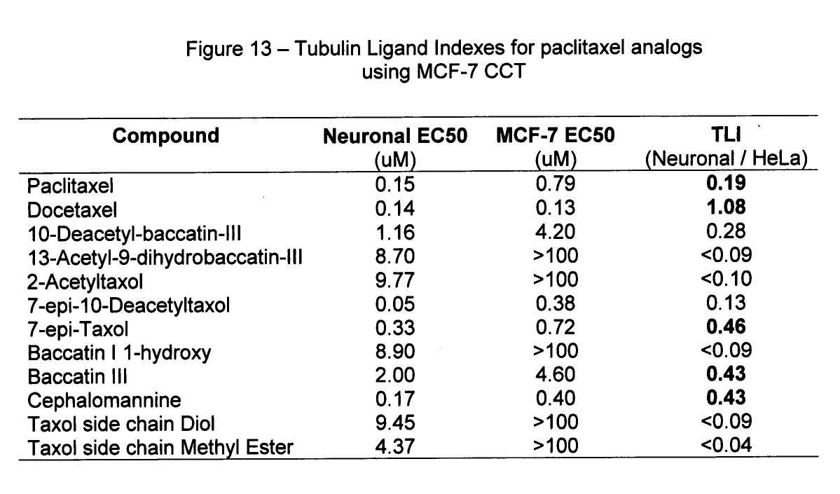

This figure describes the basic characteristics of tubulin polymerization as measured by absorbance or fluorescence. Tubulin is isolated from tissues in the form of subunit which can subsequently be induced to polymerize by manipulating temperature and buffer composition (pH, GTP and glycerol). Subunits are composed of one alpha and one β tubulin monomer to form a heterodimer (FIG. 2A). After initiation of a polymerization reaction heterodimers coalesce to form oligomeric structures called nucleating centers (see nucleation phase on Line 2 of FIG. 2B). These are the initiating points of microtubule elongation which occurs in the polymerization phase of the reaction (see Line 2). Finally the steady state phase is a balance between polymerizing and depolymerizing microtubules which result in a constant polymer mass (see Line 2). All three phases of polymerization can be affected by tubulin ligands but there are generally two basic mechanisms of action. The polymerization enhancers such as paclitaxel or epothilone B, increase the rate of nucleation, increase polymerization rate and reduce depolymerization (see Line 1). In contrast, ligands such as vinblastine and colchicine inhibit polymerization by binding to subunits which reduces their propensity to polymerize (see Line 3). Thus observing the three phases of microtubule polymerization in the presence of drug like compounds, allows one to identify and measure the potency of tubulin ligands by detecting differences from the norm.

FIG. 3: Comparison of BBT, HeLa CCT and MCF-7 CCT polymerization assays using the absorbance and fluorescent formats.

An absorbance and a fluorescent plate reader were pre-warmed to 37° C. Tubulins were resuspended to 2 mg/ml in ice cold G-PEM buffer (80 mM Pipes pH 6.9, 1 mM MgCl2, 1 mM EGTA, 10 uM DAPI and 1 mM GTP) and both formats i.e. fluorescence and absorbance measurements, were run in parallel to ensure a direct comparison. To initiate the polymerization reaction 80 ul (absorbance) or 12 ul (fluorescence) of tubulin solution was pipetted into the pre-warmed wells and the kinetic program was started. Readings were every one minute for 40 min. At 40 min samples from the absorbance measurements were centrifuged at 100,000× g at 37° C. for 30 min to pellet the microtubule polymer. Samples of pellets and supernatants were run on 10% (w/v) SDS-PAGE gels and stained with Coomassie blue dye. A indicates the absorbance data, B is the fluorescence data, where the symbols represent: In A and B—Triangles, MCF-7; Diamonds, Neuronal; Squares, HeLa. C contains the Coomassie blue stained gel of pellets, and D contains the coomassie stained gel of supernatants, where lanes 2 and 3 are neuronal tubulin, 4 and 5 are HeLa tubulin, and 6 and 7 are MCF-7 tubulin. It is noticeable that HeLa supernatants contain slightly more tubulin than neuronal or MCF-7, this finding is supported by the absorbance based polymerization assay which indicates that HeLa tubulin is still polymerizing between T=20-40 min (i.e. increasing OD values) compared to neuronal and MCF-7 tubulins which are in the typical steady state phase. The differences in the signal of polymerized tubulins in the absorbance method does not have a straightforward explanation, it is expected that equivalent concentrations of microtubules would have similar absorbance values. The explanation may reside in the width of microtubules which can vary from 9 to 14 protofilaments (16 to 25 nm), or in the length of microtubules which can alter the absorbance reading. The difference of signals in the fluorescence method are neatly explained by the different binding or quantum yields obtained from the DAPI titration in FIG. 8.

FIG. 4: Titrating glycerol concentration on tubulin polymerization.

Twenty four micrograms of BBT or CCT in G-PEM with 10 uM DAPI plus glycerol at 4° C. was pipetted into wells of the 384-well plate and incubated at 37° C. Glycerol concentrations were 0% (thick dashes), 6% (thick line), 12% (narrow dashed line), 18% (thin line) and 24% (medium thickness line) (v/v) glycerol. Tubulin polymerization was detected by measuring the fluorescence of the solution (Ex 360 nm, Em 405 nm) over time. Using these profiles, 20% glycerol was chosen as the standard amount to use for the 100% control in enhancer assays and 20% glycerol was also a good starting level for promoting polymerization in the inhibitor assays.

FIG. 5: Isolated Neuronal, HeLa and MCF-7 tubulins.

Tubulins were isolated by published procedures, neuronal (ref. 23 combined with ref. 30), HeLa and MCF-7 tubulins were isolated with a similar method to that of Bulinski and Borisy 1979 (31). Twenty micrograms of total protein were run on 8% SDS-PAGE separating gels and stained with Coomassie blue. Neuronal tubulin was greater than 99% pure whereas the cancer tubulins were >95% pure. All tubulins were stored as a lyophilized powder as described in U.S. Pat. No. 6,750,330. MAPs=Microtubule associated proteins.

FIG. 6: Measurement of β-tubulin isotype composition.

Samples of tubulin were tested for β-tubulin isotype composition. Samples of 50 or 100 ng of tubulin were run on an 8% (w/v) separating SDS-PAGE and blotted on to nitrocellulose filters. All filters were blocked in 5% (w/v) non-fat milk prior to probing with a primary anti-tubulin antibody, after 1 h incubation blots were washed in TBST three times for 10 min each, then probed with anti-IgG-HRP conjugate. Chemiluminescence detection of the antigen was documented with X-ray film exposure. Densitometry of the bands was used to calculate the percent of tubulin isotype. The reference sample was bovine brain tubulin which has been highly characterized by Banerjee and Luduena 1992 (28). B-II, β-III and β-IV are present in bovine brain tubulin in sufficient quantity to directly quantitate isotypes of tubulin in other samples. B-I tubulin is estimated based on the assuming the remainder of the tubulin, after β-II, β-III and β-IV have been determined, is composed of this isotype. This is possible because of the purity of the tubulins and accurate protein assay quantitation prior to loading exact amounts onto the gel for blotting. As a confirmation of the total amount of tubulin loaded per lane, the blots were probed with a pan-specific antibody which recognizes all tubulins uniformly (FIG. 6A, see Cytoskeleton Inc. product catalog number ATN02), compare Neuronal and MCF-7 in FIG. 6A. β-I was detected with clone SAP.4G5 monoclonal antibody (Mab) see FIG. 6B, β-II was detected with Mab clone JDR.3B8 see FIG. 6C, β-III was detected with Mab clone SDL.3D10 see FIG. 6D and β-IV was detected with Mab clone ONS.1A6 see FIG. 6E. All β-tubulin isotype MAbs were from Banerjee and Luduena 1992 (28). Secondary antibodies were anti-sheep IgG-HRP for ATN02, and anti-mouse IgG-HRP for all others. Lanes 1 and 3 contain 100 ng of purified tubulin, lanes 2 and 4 contain 50 ng of purified tubulin.

FIG. 7: Comparison of the traditional absorbance based method of measuring tubulin polymerization versus the DAPI based fluorescence assay.

A fluorescent plate reader was tuned to 360 nm excitation wavelength and 410 nm emission wavelength, a 384 well plate was placed into the machine and it was pre-warmed to 37° C. Bovine brain tubulin (Cat# TL238 from Cytoskeleton Inc.) was resuspended to 2 mg/ml in ice cold G-PEM buffer (80 mM Pipes pH 6.9, 1 mM MgCl2, 1 mM EGTA, 1 mM GTP and 10 uM DAPI). Paclitaxel analogs (LKT Inc. Cat# TT100 and TT101, docetaxel from Sigma Chemical Co. cat.# 01885) were diluted from 1 mM DMSO stocks into G-PEM buffer and 1.3 ul aliquots were pipetted into each well of the 384 well plate. To initiate the polymerization reaction 12 ul of tubulin solution was pipetted into the pre-warmed wells and the kinetic program was started. Readings were every one minute for 40 to 60 min. The Vmax was taken from the polymerization phase of each graph and this reading was compared to a 20% (v/v) glycerol containing reaction which was defined as 100 units or 100% (see FIG. 7A). Some values are greater than 100% because the paclitaxel analogs polymerize tubulin greater than 20% glycerol in a reaction. Other data comparisons are using 1/EC50 (B) and AFU or absorbance at T=20 min (C). Symbols represent: Open diamond, 13-Acetyl-9-dihydrobaccatin-III; Open square, 2-Acetyltaxol; Closed triangle, 7-epi-10-deacetyltaxol; Closed diamond, 7-epi-Taxol; Star, Baccatin III; Open circle, Baccatin III; Closed circle, Cephalomannine; Closed square, Paclitaxel; Open triangle, Taxol side chain Diol; Dash, Taxol side chain Methyl Ester; Plus sign, 10-Deacetyl-baccatin-III.

FIG. 8: Titration of DAPI in the presence of BBT, HeLa and MCF-7 CCT.

Bovine brain tubulin (mainly isotypes βII and βIII) has a Kd (affinity constant) for DAPI of 6 uM in the polymer form and 43 uM in the monomer form (24). We measured the Kd for DAPI by titrating the DAPI from 1 nM to 200 uM in the presence of 1 mg/ml (10 uM) BBT, HeLa CCT and MCF-7 CCT in G-PEM buffer. The resultant fluorescent values are plotted on the graph. Symbols represent: Diamonds, BBT; Triangles, HeLa CCT; Square, MCF-7 CCT.

You will notice that the BBT line is similar to that reported by Bonne et al. 1985 (24), i.e. a saturation type curve which peaks at 100 uM DAPI. Similarly MCF-7 CCT has a saturation curve which peaks at 100 uM although this curve has a higher quantum fluorescence yield at all concentrations compared to the BBT sample. In contrast the CCT line from HeLa tubulin is biphasic (0 to 10 uM and 10 to 200 uM), this represents the presence of two species of different affinity. This may represent the proportion of tubulin isotypes in this tubulin which is 10% βIV and 90% βI. Therefore we assume that βI tubulin binds DAPI with low affinity whereas βIV (like βII and βIII) binds with high affinity. This is backed-up by corresponding data in FIGS. 3,4,9 where HeLa CCT has much lower fluorescence when fully polymerized indicating again that βI has much lower affinity for DAPI.

FIG. 9: Inhibitor and enhancer example polymerization assays using BBT, HeLa CCT and MCF-7 CCT in the presence of colchicine, vinblastine and paclitaxel.

Compounds were assayed in the standard glycerol containing (vinblastine and colchicine) or no glycerol containing (paclitaxel) formats. Paclitaxel analogs were from LKT Laboratories Inc. Vinblastine sulphate was from Ceres Chemical Inc. and colchicine was from Sigma Chemical Company. Twenty four micrograms of CCT in G-PEM with 10 uM DAPI plus glycerol (colchicine and vinblastine) or minus glycerol (paclitaxel) at 4° C. was pipetted into wells of the 384-well plate which contained 1.3 ul of 10× strength compound concentration and incubated at 37° C. Tubulin polymerization was detected by measuring the fluorescence of the solution (Ex 360 nm, Em 405 nm) over time. Each set of data, A through I, represents six duplicate polymerization assays from one experiment, the duplicates contain either a 20% glycerol control or one of five compound concentrations (0.1, 0.3, 1.0, 3.0 or 10 uM). Paclitaxel experiments were repeated n=3 for neuronal, n=5 for HeLa and n=2 for MCF-7. Vinblastine experiments were repeated n=2 for neuronal, n=5 for HeLa and n=2 for MCF-7. Colchicine experiments were repeated n=2 for neuronal, HeLa and MCF-7. In A,B and C the compound is paclitaxel, the 20% glycerol control reaction is represented by Open Diamonds. In A,B and C the other symbols are: Closed Squares, 0.1 uM; Open Squares, 0.3 uM; Closed Diamonds, 1.0 uM; Open Triangles, 3.0 uM; Closed Triangles, 10 uM. In D,E and F the compound is vinblastine, the 20% glycerol control reaction is represented by Closed Diamonds. In D,E and F the other symbols are: Closed Squares, 0.1 uM; Closed Triangles, 0.3 uM; Open Squares, 1.0 uM; Open Triangles, 3.0 uM; Open Diamonds, 10 uM. In G,H and I the compound is colchicine, the 20% glycerol control reaction is represented by Closed Diamonds. In G,H and I the other symbols are: Closed Squares, 0.1 uM; Closed Triangles, 0.3 uM; Open Squares, 1.0 uM; Open Triangles, 3.0 uM; Open Diamonds, 10 uM. Neuronal tubulin is represented in A,D and G. HeLa tubulin is represented in B,E and H. MCF-7 tubulin is represented in C,F and I.

FIG. 10: Dose response curves for paclitaxel, vinblastine and colchicine.

Data from FIG. 9 is reduced to a rate “Vmax” in fluorescence units per minute, then the inhibition or enhancement activity is calculated as a percentage of the “20% glycerol control reaction”'s Vmax. These values are plotted against the log10 concentration of drug, and the 50% inhibition or enhancement drug concentration is read off the x-axis for IC50 or EC50 respectively. Symbols represent: Squares, Neuronal; Triangles, HeLa; Diamonds, MCF-7.

FIG. 10A represents paclitaxel, B represents vinblastine and C represents colchicine.

FIG. 11: Calculation of the Tubulin Ligand Index (TLI) ratio for BBT, HeLa CCT and MCF-7 CCT in the presence of paclitaxel, vinblastine and colchicine.

The IC50 or EC50 values are compared to neuronal tubulin values using the Tubulin Ligand Index (TLI) ratio method (this patent). BBT (neuronal) values are divided by CCT (cancer) values to obtain the TLI ratio. For compounds selectively targeting BBT the TLI would be less than 1.0, whereas for drugs selectively targeting CCT the values would be greater than 1.0.

FIG. 12: Tubulin Ligand Indexes for paclitaxel analogs using HeLa CCT.

Using the conditions described in FIG. 9, we measured twelve taxol analogs against BBT and HeLa CCT. TLI's were calculated by the method described in FIG. 11. The four TLI values in bold represent the highest values in the set which target HeLa CCT more effectively than the other analogs. Results represent the EC50's obtained at 2 mg/ml tubulin.

FIG. 13: Tubulin Ligand Indexes for paclitaxel analogs using MCF-7 CCT.

Using the conditions described in FIG. 9, we measured eleven taxol analogs against BBT and MCF-7 CCT. TLI's were calculated by the method described in FIG. 11. The four TLI values in bold represent the highest values in the set which target HeLa CCT more effectively than the other analogs. Results represent the EC50's obtained at 2 mg/ml tubulin.

### METHODS

Compound Preparation

Paclitaxel analogs were purchased from LKT Labs Inc. in 8-well strips at 1 mM compound in DMSO. These stocks were diluted into G-PEM buffer to 10× concentrations prior to pipetting 1.3 ul into each well. Vinblastine was purchased from Ceres Chemical Inc. as a powder, a 1 mM stock was made in DMSO and dilutions were made into G-PEM buffer. Colchicine was purchased from Sigma Chemical Co. The compound stocks are prepared at 1, 3, 10, 30 and 100 uM in G-PEM at RT. The 20%-glycerol reaction is considered the control reaction for IC50s and EC50s (Defined as 100% Vₘₐₓ).

Tubulin Isolations

Tubulins were isolated by published procedures. Bovine brain tubulin (BBT) is isolated as described by Shelanski et al. 1973 (23) in combination with ionic exchange removal of MAP proteins as described by Lee and Timasheff 1976 (30). Essentially, Shelanski et al 1973 purified active polymerizable tubulin by homogenizing brain tissue in 80 mM Mes pH 6.5, 1 mM GTP, 1 mM MgCl2 and 1 mM EGTA and then centrifuging to pellet denatured protein, this was followed by warming the supernatant to 37° C. to induce microtubule polymerization. The polymers which constitute the active protein, are purified by sedimentation at 100,000× g. The pellet is resuspended in the same buffer and the tubulin is polymerized again. The second sedimented polymer pellet is resuspended in 50 mM MES buffer pH 6.5 and it is passed over a phospho-cellulose column to remove MAPs which results in a >99% purified tubulin.

HeLa and MCF-7 tubulins were isolated with a similar method to that of Bulinski and Borisy 1979 (31). Essentially, as for neuronal tubulin the polymers are purified by centrifugation at 100,000× g and then passed over a PC column to remove MAP proteins. Even though the PC column is employed some MAPs remain and are present in the final preparation (see FIG. 5), the final preparation is >95% pure tubulin. The samples are treated for lyophilization as described in U.S. Pat. No. 6,750,330.

Equipment

A temperature controlled fluorescence plate reader is used which measures fluorescence in 384-well plates at Excitation of 360 nm±20 nm and Emission 405 nm±10 nm. Emission wavelengths higher than this can be used because DAPI fluoresces with a maxima of 450 nm with shoulders out to 400 and 480 nm. The program must be capable of storing readings every minute for 60 min.

Basic Assay Format

A fluorescent plate reader is tuned to 360 nm excitation wavelength and 405 to 475 nm emission wavelength, a 384 well plate was placed into the machine and it was pre-warmed to 37° C. BBT (Cat# TL238 from Cytoskeleton Inc.) or CCT (either Catalog number H001 [HeLa] or H005 [MCF-7] from Cytoskeleton Inc.) was resuspended to 2 mg/ml in ice cold G-PEM buffer (80 mM Pipes pH6.9, 1 mM MgCl2, 1 mM EGTA, 1 mM GTP and 10 uM DAPI)(enhancer assays) or ice cold G-PEM plus 20% glycerol (inhibitor assays). Paclitaxel analogs (LKT Inc. Cat# TT100 and TT101) were diluted from 1 mM DMSO stocks into G-PEM buffer and 1.3 ul aliquots were pipetted into each well of the 384 well plate. To initiate the polymerization reaction 12 ul of tubulin solution was pipetted into the pre-warmed wells and the kinetic program was started. Readings were every one minute for 40 to 60 min. The Vmax was taken from the polymerization phase of each graph and this reading was compared to a 20% (v/v) glycerol containing reaction which was defined as 100 units or 100%. Some values are greater than 100% because the paclitaxel analogs polymerize tubulin faster than 20% v/v glycerol.

Data Reduction and Analysis for IC50.

Data is reduced to V10 (velocity at T=10 min) using the plate reader software and converted into the percent of the 20%-glycerol-control's V10, i.e. using the zero compound concentration wells. A plot of log10 of concentration (X-axis) and Percent Vmax (control) (Y-axis) is made for each compound. The intercept of 50% inhibited (IC50) velocity is the log10 of the IC50 value.

Data Reduction and Analysis for EC50.

Data is reduce to Vmax (maximum slope of the polymerization phase) using the plate reader software. The Vmax data is converted into percent of the 20%-glycerol-control's Vmax. Then the log10 of concentration is plotted on the X-axis and Percent Vmax (control) on the Y-axis for each compound. The intercept of the 50% enhanced (EC50) velocity is the log10 of the EC50 value.

### EXPERIMENTAL RESULTS

Purification of Cancer Cell Tubulins and Analysis of Their Isotypic Composition

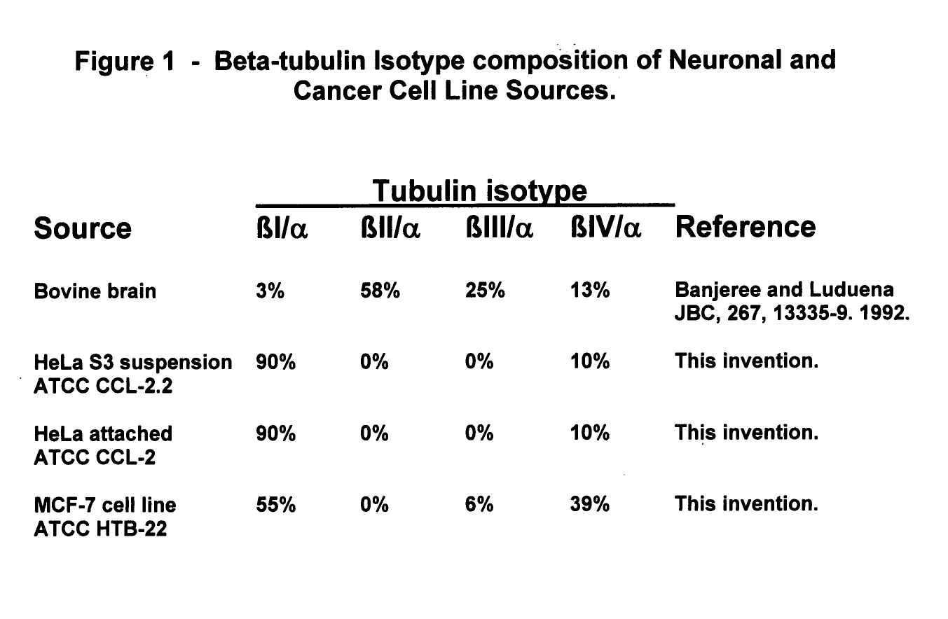

Tubulins were isolated by published procedures, (23,30,31), to >99% pure (BBT) or >90% pure (CCTs) see FIG. 5). Samples of tubulin were tested for β-tubulin isotype composition by western analysis. Samples of 50 and 100 ng were run on an 8% (w/v) separating SDS-PAGE and blotted on to nitrocellulose filters. The protein loading was checked comparing BBT and CCT on the same gels (see FIG. 6) by probing with an anti-tubulin pan-specific antibody (Cytoskeleton Inc. Cat# ATN02). FIG. 6a, shows that there were equal loadings of tubulin protein in the 50 ng lanes and in the corresponding 100 ng lanes of different tubulins. This exact protein loading enabled direct comparison between different tubulins, and using specific isotype antibodies and BBT as an established standard of tubulin isotypes (see FIG. 1 and 28), we were able to directly calculate nanogram quantities of isotypes in the CCT. Only αβII, αβIII and αβIV could be quantitated in this way, al was not present in BBT and therefore because it was abundant in CCT (see FIG. 6b) we assumed it made up the remainder of the tubulin in cancer cells. Obviously there could be αβV and αβVI isotypes but these are known to be very minor components in most cell types (15, 34), and in addition xenografts of ovarian cancer are known to contain 70-80% of the βI isotype and much less of other isotypes (Katsetos et al. 2002), so we are confident these percentage calculations represent a realistic estimate of the isotypic composition.

Typical Tubulin Polymerization Data

FIG. 2 describes the basic characteristics of tubulin polymerization as measured by absorbance or fluorescence. Tubulin is isolated from tissues in the form of subunit which can subsequently be induced to polymerize by manipulating temperature and buffer composition (pH, GTP and glycerol). Subunits are composed of one alpha and one β tubulin monomer to form a heterodimer. After initiation of a polymerization reaction heterodimers coalesce to form oligomeric structures called nucleating centers (see Nucleation phase in FIG. 2). These are the initiating points of microtubule elongation which itself occurs in the polymerization phase of the reaction (see Polymerization phase in FIG. 2). Finally the steady state phase is a balance between polymerizing and depolymerizing microtubules which result in a constant polymer mass. All three phases of polymerization can be affected by tubulin ligands but there are generally two basic mechanisms of action. The polymerization enhancers such as paclitaxel or epothilone B, increase the rate of nucleation, increase polymerization rate and reduce depolymerization (see Line 1 in FIG. 2). In contrast, ligands such as vinblastine and colchicine inhibit polymerization by binding to subunits which reduces their propensity to polymerize (see Line 3 in FIG. 2). Thus observing the three phases of microtubule polymerization in the presence of drug like compounds, allows one to identify and measure the potency of tubulin ligands by detecting differences from the norm.

Validating the Fluorescence Based Assay Format

We compared eleven paclitaxel analogs for their ability to affect polymerization of bovine brain tubulin in the absorbance and fluorescence modes of detection. Their EC50s were compared on a scatter plot which allows the linearity of the relationship to be assessed (see FIG. 7). As you can see from the graph both modes of detection could identify the derivatives with polymerization promoting activity. Their relationship was curvi-linear with the fluorescent method of detection being the most sensitive. This comparison allowed us to proceed with confidence that the fluorescence method could be utilized in a miniature 10-12 ul format for measuring polymerization of cancer cell tubulin.

The assay utilizes a fluorescent compound which binds to tubulin and microtubules (DAPI, ref: Bonne et al. 1985). The quantum yield of fluorescence is increased when the fluorophore is bound to the tubulin and microtubules alike, however there is a ten-fold increase in the affinity of the fluorophore for microtubules compared to tubulin. The result is a fluorescence signal that closely follows microtubule formation. Indeed, using bovine neuronal tubulin to compare IC50 values in the fluorescence assay versus the traditional absorbance based assay, the fluorescent microassay was within 20% of the value of the traditional absorbance assay. One caveat that must be recognized is that the site of fluorophore binding may overlap with your compound's binding site thereby interfering with the detection of polymer and affecting the IC50 determination. This has not been the case for three well known binding sites of tubulin (colchicine, paclitaxel and vinblastine) which are not affected when bovine neuronal tubulin is used in both formats.

Validating the Tubulin Ligand Index with Known Anti-Cancer Drugs

Using cancer cell line tubulin to generate the dose response curves (see FIG. 9), the assay has been used to determine the EC50 or IC50 of two known tubulin ligands, namely paclitaxel and vinblastine. In the table, the results are compared with bovine brain tubulin using the same microassay. A ratio, called the Tubulin Ligand Index ratio (TLI ratio=IC50 bovine brain tubulin divided by the IC50 cancer cell line tubulin) compares the bovine brain tubulin with the cancer cell line tubulin. These results indicate that this new microassay may be used to compare known compounds for their efficacy on cancer and neuronal tubulins.

We also tested twelve paclitaxel analogs against neuronal, HeLa and MCF-7 tubulin. As FIGS. 12 and 13 show the cancer cell tubulins were similarly affected or less affected by these analogs than neuronal tubulin. This is surprising because paclitaxel is used for treating cancer, but this finding may explain the neurotoxic effects of the paclitaxel family. Within the analog set there is variation of their efficacy as measured by the TLI, i.e. values range from <0.01 to 0.93 for HeLa TLIs, whereas MCF-7 TLIs range from <0.04 to 1.08. Also, the first three lines of data on both tables indicate that the TLI values are specific for a particular type of cancer tubulin, i.e. 10-Deacetyl-baccatin-III's TLI is <0.01 for HeLa but 0.28 for MCF-7. Therefore all cancer tubulins cannot be pooled as “cancer tubulin”, each specific cancer tubulin has its own character which can be exploited in the TLI analysis. Using the TLI system for analysis of analog sets is particularly powerful in drug discovery because the individual members are directly compared.

As an example of a high TLI within an analog set, we identified docetaxel as having the highest TLI for HeLa cancer tubulin (TLI=0.93). In the clinic this compound is less neurotoxic than paclitaxel (ref. 3, Ch. 2, p 131). In fact the lower toxicity of docetaxel is probably related to the lower dose required compared to paclitaxel. For example, for 3 week interval intravenous infusions the dosage is 80 mg/m² for docetaxel compared to 160 mg/m² for paclitaxel. As Table 12 shows, docetaxel's EC50 using HeLa CCT is four fold lower (i.e. more potent) than paclitaxel's EC50 (i.e. 0.15 uM compared to 0.79 uM), this is in contrast to the EC50 values obtained using neuronal tubulin which are very similar (i.e. 0.14 uM compared to 0.15 uM). Thus the difference in TLI between paclitaxel and docetaxel can be attributed to the increased affect of docetaxel on HeLa CCT. The results indicate that an increase in TLI at the paclitaxel binding site may result in an improved therapeutic index.

An example of a high TLI value in a pre-clinical compound: Disorazol E1 from Zentaris GmbH. Using the conditions described in FIG. 9, Zentaris GmbH measured Disorazol E1 (vinblastine analog) against BBT and HeLa CCT. IC50 were estimated from the 50% intercept of a dose response curve. TLI's were 1.0 for vinblastine and 2.0 for Disorazol E1 (32), indicating that Disorazol E1 is twice as effective as vinblastine at targeting CCT compared to BBT. Clinical trials should tell whether this compound is less neurotoxic than vinblastine as the TLI values suggest.

The Tubulin Ligand Index System

The Tubulin Ligand Index system is different from previous analysis of tubulin isotypes and cancer tubulin sources (e.g. 16,26,27,28,29) because it creates a measurement that is able to be cross referenced between experiments. By comparing the IC50 or EC50 values between two tubulins, one of pathogenic origin and one of normal tissue origin, an exact ratio of specificity can be determined. The key step in standardizing this measurement is to use glycerol as a polymerization enhancing agent for example in the “20% v/v glycerol control reaction”. Glycerol is known to enhance tubulin polymerization by the exclusion of water molecules from sites of protein:protein association (33). So glycerol can be viewed as a global enhancer of polymerization in contrast to tubulin ligands that target one site on the molecule. Thus using 20% v/v glycerol we can standardize every assay no matter what source of tubulin or molecule being tested. It is clear that other standardizing agents could be used (e.g. DMSO, sucrose or other glycerol concentration) in place of 20% v/v glycerol.

An additional powerful aspect of the TLI system is it's independence on the exact concentration of drug. In the construction of chemical libraries for drug screening where thousands of compounds are weighed out prior to making solutions, mistakes are made during calculation of each compound's molarity and the weighing process, which creates erroneous drug concentrations. If TLI results are obtained when using the same stock solution of a compound, the calculation of a ratio means that upto ten fold errors in drug concentration can be tolerated. This is due to both tubulins being exposed to the same concentration range which cancels out concentration errors when one IC50 is divided by the other.

Importance to Future Drug Discovery Efforts

The relationship between a compounds activity on neuronal versus cancer cell tubulin is at the root of this invention, this comparison can only be achieved by rigorous comparative analysis of the activity of compounds on both tubulins. For example, Compound X with an IC50 of 5 uM for CCT, and 5000 uM for BBT giving a TLI=1000 could be more efficacious in the clinic than Compound Y which has an IC50 of 1 uM, i.e. higher affinity for CCT, but an IC50 of 100 uM for BBT which is only a of TLI=100.

The fact that there are known IC50 values for a vast number of drugs tested against BBT means that by using one concentration of compound in a screen e.g. 1 uM or 10 uM it is possible to select the compounds that are particularly active against CCT with just one reaction. This approach would save on the expensive CCT protein and make larger screens more economical. In a similar vein the SPA tubulin ligand assay described in the Background section can be used as an alternative economical format to measure ligand binding to a particular site, thus a TLI value can be obtained from this assay as well.

We believe the importance of αβI or the actual mixture of tubulins in a cell has been overlooked because the major source of tubulin in the past has been mammalian or avian brain tissue which has mainly αβII and αβIII which does not represent the isotypic composition of many cancer cell tubulins. In addition, it has recently been shown that the βI tubulin isotype is important for cell motility (35). Therefore a screen, such as the one described here for HeLa tubulin which contains 90% βI tubulin, will be important for developing drugs that can inhibit cell motility such as occurs in metastatic cancers.

It has been reported that βIII tubulin isotype predominates in late stage and paclitaxel resistant tumors (17,18). Therapeutic regimes for cancer treatment can be devised based on targeting βI tubulin isotype during initial stages when the cancer cells are similar to the surrounding tissue of origin. However in medium and later stages of cancer development the isotype composition may change because there is selective pressure against the βI tubulin or by resistance to paclitaxel as stated above. In this case the medium and late stage cancers can be targeted with anti-βIII tubulin ligands.

Another possibility is to target mutated β-tubulins that have been detected in patients (e.g. U.S. Pat. No 6,251,682). Isolation of mutant tubulins can be accomplished by using tissue culture created clones that express a particular mutated β-tubulin gene, these cells can be used as a source of mutant tubulin (e.g. ref. 29).

Other cell line tubulins will clearly be a source of great interest for additional drug targeting specificity, i.e. colon or liver cancer cells, fibrosarcoma, glioblastoma etc. These pathogen originating tubulins would be compared to tubulin from a standard tissue or cell type that shows the most cytotoxicity to currently used anti-tubulin drugs e.g. neuronal tubulin for neurotoxcity. Also fungal cell tubulin compared to mammalian or plant tubulins would give indications of specificity for killing fungal pathogens, and similarly yeast, nematode, trypanosomes and helminth tubulins would be good targets for developing drugs to cure these diseases, this will be the focus of the next application.

### REFERENCES

Patents
U.S. Pat. No. 6,750,330 Lyophilized tubulins.
U.S. Pat. No. 6,512,003 Synthetic spiroketal pyranes as potent anti-cancer compounds and use.
U.S. Pat. No. 6,500,405 Use of certain amides as probes for detection of anti-tubulin activity and resistance monitoring.
U.S. Pat. No. 6,346,389 Methods of selectively modulating the interactions between survivin and tubulin.
U.S. Pat. No. 6,329,420 Tubulin binding compounds (COBRA).
U.S. Pat. No. 6,306,615 Detection method for monitoring P tubulin isotype specific modification.
U.S. Pat. No. 6,251,682 Methods and markers for prognosticatin efficacy of anticancer agents.
U.S. Pat. No. 5,972,640 Methods for identifying antimitotic agents.
U.S. Pat. No. 5,965,718 Analogs of sarcodictyin and eleutherobin.
U.S. Pat. No. 5,760,092 Allocolchinones and uses thereof.

Articles
1. Levine A S Ed., 1982, Cancer in the young, Masson Publishing, New York
2. Teicher B A Ed., 1997, Cancer Therapeutics: Experimental and Clinical Agents, Humana Press, Totowa, N.J.
3. Chu E. and DeVita V T. 2004. Physician's Cancer Chemotherapy Drug Manual. Jones and Bartlett Publishers, Sudbury, Mass., USA.
4. George P, Journey L J and Goldstein M N. 1965. Effect of vincristine on the fine structure of HeLa cells during mitosis. J. Natl. Cancer Inst. 35, 355.
5. Schiff P B, Fant J and Horwitz S B. 1979. Promotion of microtubule assembly in vitro by Taxol. Nature 22, 665-667.
6. Smith C D, Zhang X, Mooberry S L, Patterson G M. and Moore R E. 1994. Cryptophycin: A new anti-microtubule agent active against drug-resistant cells. Cancer Research, 54(14), 3779-84.
7. Chen B D, Nakeff A. and Valeriote F. 1998. Cellular uptake of a novel cytotoxic agent, cryptophycin-52, by human THP-1 leukemia cells and H-125 lung tumor cells. International Journal of Cancer, 77(6), 869-873.
8. Pettit G R, Kamano Y, Fujii Y, Herald C L, Inoue M, Brown P, Gust D, Kitshara K, Schmidt J M, Doubek D L. and Michel C. 1981. Marine animal biosynthetic constituents for cancer chemotherapy. Journal of Natural Products, 44(4), 482-5.
9. Bai R, Pettit G R. and Hamel E. 1990. Dolastatin 10, a powerful cytostatic peptide derived from a mrine animal. Inhibition of tubulin polymerization mediated through the vinca alkaloid binding domain. Biochemical Pharmacology, 39(12), 1941-9.
10. Edelman M J, Gandara D R, Hausner P, Israel V, Thornton D, DeSanto J. and Doyle L A. 2003. Phase 2 study of cryptophycin 52 (LY355703) in patients previously treated with platinum based chemotherapy for advanced non-small cell lung cancer. Lung Cancer, 39(2), 197-9.
11. Stevenson J P, Sun W, Gallagher M, Johnson R, Vaughn D, Schuchter L, Algazy K, Hahn S, Enas N, Ellis D, Thornton D, and O'Dwyer P J. 2002. Phase 1 trial of the cryptophycin analogue LY355703 administered as an intravenous infusion on a day 1 and 8 schedule every 21 days. Clinical Cancer Research, 8(8), 2524-9.
12. Saad E D, Kraut E H, Hoff P M, Moore D F Jr, Jones D, Pazdur R. and Abbruzzese J L. 2002. Phase II study of dolastatin 10 as first line treatment for advanced colorectal cancer. American Journal of Clinical Oncology, 25(5), 451-3.
13. Kerbrat P, Dieras V, Pavlidis N, Ravaud A, Wanders J, and Fumoleau P. 2003. Phase II study of LU 103793 (dolastatin analog) in patients with metastatic breast cancer. European Journal of Cancer, 39(3), 317-20.
14. Marks R S, Graham D L, Sloan J A, Hillman S, Fishkoff S. Krook J E, Okuno S H, Maillard J A, Fitch T R. and Addo F. A phase II study of the dolastatin 15 analogue LU 103793 in the treatment of advanced non-small cell lung cancer. American journal of Clinical Oncology, 26(4), 336-7.
15. Verdier-Pinard P, Wang F, Martello L, Burd B, Orr G A and Horwitz S B. 2003. Analysis of tubulin isotypes and mutations from taxol-resistant cells by combined isoelectrofocusing and mass spectrometry. Biochemistry, 42, 5349-5357.
16. Sackett D L, Giannakakou P, Poruchynsky M and Fojo A. 1997. Tubulin from paclitaxel-resistant cells as a probe for novel antimicrotubule agents. Cancer Chemotherapy and Pharmacology, 40 (3), 228-232.
17. Banerjee A. 2002. Increased levels of tyrosinated alpha-, β(III)-, and β(IV)-tubulin isotypes in paclitaxel-resistant MCF-7 breast cancer cells. Biochemical and Biophysical Research Communications, 293, 598-601.
18. Katsetos C D, Del Valle C, Geddes J F, Aldape K, Boyd J C, Legido A, Kahlili K, Perentes E. and Mork S J. 2002. Localization of the neuronal class III b-tubulin in oligodendrogliomas: Comparison with Ki-67 proliferative index and 1p/19q status. Journal of Neuropathology and Experimental Neurology, 61, 307-320.
19. Nicoletti M I, Valoti G, Giannakakou P, Zhan Z, Kim J H, Lucchini V, Landoni F, Mayo J G, Giavazzi R, and Fojo T. 2001. Expression of beta-tubulin isotypes in human ovarian carcinoma xenografts and in a sub-panel of human cancer cell lines from the NCI-Anticancer Drug Screen: Correlation with sensitivity to microtubule active agents. Clinical Cancer Research, 7(9), 2912-22.
20. Puck T T, Marcus P I, and Cieciura S J. 1955. A rapid method for viable cell titration and clone production with HeLa cells in tissue culture: the use of x-irradiated cells to supply conditioning factors. Proceedings of the National Academy of Science, 41, 432-437.
21. Puck T T, Marcus P I, and Cieciura S J. 1956. J. Exp. Med. 103, 273-284.
22. Tahir S K, Kovar P, Rosenberg S H, and Ng S-C. 2000. Rapid colchicine competition-binding scintillation proximity assay using biotin-labeled tubulin. Biotechniques, 29, 156-160.
23. Shelanski M L, Gaskin F. and Cantor C R, 1973. Proceedings of the National Academy of Science, 70, 765-768.
24. Bonne D, Heusele C, Simon C. and Pantaloni D. 1985. 4′,6-Diamidino-2-phenylindole, a fluorescent probe for tubulin and microtubules. Journal of Biological Chemistry, 280 (5), 2819-2825.
25. Avila, J. Ed. 1990. Microtubule Proteins. CRC Press, Boca Raton, Fla.
26. Derry W B, Wilson L, Khan I A, Luduena R F. And Jordan M A. 1997. Taxol differentially modulates the dynamics of microtubules assembled from unfractionated and purified β-tubulin isotypes. Biochemistry. 36, 3554-3562.
27. Lobert S, Frankfurter A, Correia J J, 1998. Energetics of vinca alkaloid interactions with tubulin isotypes. Cell Motility and the Cytoskeleton, 39, 107-121.
28. Banerjee A. and Luduena R F. 1992. Kinetics of colchicine binding to purified β-tubulin isotypes from bovine brain. Journal of Biological Chemistry, 267, 13335-13339.
29. Giannakakou P, Sackett D L, Kamg Y K, Zhan Z, Buters J T, Fojo T. and Poruchynsky M S. 1997. Paclitaxel-resistant human ovarian cancer cells have mutant β-tubulins that exhibit impaired paclitaxel driven polymerization. Journal of Biological Chemistry, 272 (27), 17118-25.
30. Lee J C. and Timasheff S N, 1977. In vitro reconstitution of calf brain microtubules: effects of solution variable. Biochemistry, 16, 1754-1762.
31. Bulinski J C. and Borisy G G, 1980. Self-assembly of microtubules in extracts of cultured HeLa cells and the identification of HeLa microtubule-associated proteins. Proceedings of the National Academy of Science, 76 (1), 293-7.
32. Baasner S, Gerlach M, Hoefle G, Mueller G, Paulini K, Polymeropoulos E, Sasse F, Schmidt P and Guenther E G. 2003. Proceedings of the American Association for Cancer Research, 44, 2^{nd} Ed, p. 532, Abstract # 2717.
33. Gekko K. and Timasheff S N, 1981. Thermodynamic and kinetic examination of protein stabilization by glycerol. Biochemistry, 20, 4677-4685.
34. Arai K, Maruo K, Ara K Y, Uehara K. and Matsuda H. 2001. Characterization of isotype-specific regions of five classes of canine β-tubulin and their expression in several tissues and cell cultures. Journal of Veterinary Medical Science, 63 (12), 1297-1302.
35. Lezama R, Castillo A, Luduena R F and Meza I. 2001. Over-expression of β I tubulin in MDCK cells and incorporation of exogenous β I tubulin into microtubules interferes with adhesion and spreading. Cell Motility and the Cytoskeleton, 50 (3), 147-60.
36. Jaffrezou J P, Dumontet C, Derry W B, Duran G, Chen G, Tsuchiya E, Wilson L, Jordan M A, and Sikic B I. 1995. Oncology Research, 7, 517-527.
37. Ranganathan S, Dexter D W, Beneratos C A, Chapman A F, Tew K D. and Hudes G R. 1996. Cancer Research, 56, 2584-2589.
38. Davis A S, Sage R S, Wilson L, and Farrell K W. 1993. Purification and biochemical characterization of tubulin from the budding yeast Saccharomyces cerevisiae. Biochemistry, 32, 8823-8835.

1. A system using a microassay which measures the activity of a tubulin ligand on polymerizing mammalian brain and cancer cell tubulin simultaneously by calculation of the Tubulin Ligand Index, so that a comparison can be made as to the potency of the compound towards cancer cell tubulin and hence efficacy toward cancer treatments.

2. A system such as that described in claim 1, where the cancer tubulin is isolated from MCF-7 cells.

3. A system such as that described in claim 1, where the cancer tubulin is isolated from HeLa cells.

4. A system such as that described in claim 1, where the cancer tubulin is isolated from other cancer cell lines or cancer tissue.

5. A system such as that described in claim 1, where the cancer tubulin is made from a recombinant source in non-cancer cells (e.g. bacteria, yeast, fungal or insect), other cancer cells or a non-cell based protein expression system.

6. A system such as that described in claim 1, where the cancer tubulin isotypes are replaced with similar tubulin isotypes from other tissues or cells.

7. Using the system described in claim 1 to develop anti-cancer drugs that target any pathogenic origin tubulin with greater avidity than other control “normal” tubulin sources.

8. Using the system described in claim 1 to develop anti-fungal drugs that target fungal tubulin more affectively than a human or mammalian tubulin.

9. Using the system described in claim 1 to develop anti-cancer drugs that target any tubulin isotype.

10. Using the system described in claim 1 to develop anti-cancer drugs that target β I tubulin isotype.

11. Using the system described in claim 1 to develop anti-cancer drugs that target β II tubulin isotype.

12. Using the system described in claim 1 to develop anti-cancer drugs that target β III tubulin isotype.

13. Using the system described in claim 1 to develop anti-cancer drugs that target β IV tubulin isotype.

14. Using the system described in claim 1 to develop anti-cancer drugs that target β V tubulin isotype.

15. Using the system described in claim 1 to develop anti-cancer drugs that target β VI tubulin isotype.

16. Using biotinylated mammalian brain and cancer cell tubulins in the scintillation proximity format to achieve results that are similarly useful at identifying and measuring tubulin ligand activity in a similar way as the polymerization assay results are used as described in claim 1.

17. Use of the DAPI fluorescence tubulin polymerization assay in the analysis of polymerization of HeLa cell derived tubulins.

18. Use of the DAPI fluorescence tubulin polymerization assay in the analysis of polymerization of MCF-7 cell derived tubulins.

19. Use of the DAPI fluorescence tubulin polymerization assay in the analysis of polymerization of cancer cell tubulins.

20. Use of a specific concentration of compound, preferably 1 nM to 1 mM, more preferably 100 nM to 50 uM and most preferably between 1 uM and 10 uM, for screening purposes against CCT that makes screening more economical.

21. The Tubulin Ligand Index system for measuring the specificity of anti-cancer or anti-pathogen tubulin ligands.

22. A system such as that described in claim 1, where the microassay is between 1 femtoliter and 10 milliliter in volume, but preferentially less than 200 microliter, more preferentially less than 50 microliter, most preferentially less than 15 microliter.

23. Using the system described in claim 14 to develop anti-cancer drugs that target any tubulin isotype.

24. Using the system described in claim 14 to develop anti-cancer drugs that target β I tubulin isotype.

25. Using the system described in claim 14 to develop anti-cancer drugs that target β II tubulin isotype.

26. Using the system described in claim 14 to develop anti-cancer drugs that target β III tubulin isotype.

27. Using the system described in claim 14 to develop anti-cancer drugs that target β IV tubulin isotype.

28. Using the system described in claim 14 to develop anti-cancer drugs that target β V tubulin isotype.

29. Using the system described in claim 14 to develop anti-cancer drugs that target β VI tubulin isotype.

30. Using the system described in claim 1 with Vmax data reduction.

31. Using the system described in claim 1 with Absorbance or Fluorescence measurements at a particular time point in the reaction e.g. 10 or 20 min.

32. Using the system described in claim 1 with a mixture of Vmax, nucleation or measurements at particular time points or maximum obtained values.
